(19) United States
(12) Patent Application Publication
(10) Pub. No.: US 2026/0170691 A1
(43) Pub. Date: Jun. 18, 2026

(54) METHOD FOR DETERMINING AN INSTANTANEOUS DEPTH OF FOCUS OF A USER OF SMART GLASSES, AND SMART GLASSES

(71) Applicant: Robert Bosch GmbH, Stuttgart (DE)

(72) Inventors: Alexander Zimmer, Stuttgart (DE); Johannes Meyer, Haseluenne (DE)

(21) Appl. No.: 19/128,658

(22) PCT Filed: Jan. 29, 2024
(86) PCT No.: PCT/EP2024/052062

§ 371 Date: May 9, 2025

(30) Foreign Application Priority Data
Feb. 10, 2023 (DE) 10 2023 201 093.3

(51) Int. Cl. G06T 7/80; A61B 3/00; A61B 3/11; A61B 3/113; A61B 3/14; G06F 3/01

(52) U.S. Cl. CPC G06T 7/80; A61B 3/0025; A61B 3/111; A61B 3/113; A61B 3/14; G06F 3/013

(57) ABSTRACT

A method for determining an instantaneous depth of focus of a user of smart glasses, using a single-eye tracking system. In the method, an instantaneous Listing plane of a user eye is measured. An instantaneous vergence of the user eye is ascertained on the basis of the measured instantaneous Listing plane of the user eye. The user-specific instantaneous depth of focus of the user eye is ascertained from the ascertained instantaneous vergence of the user eye.

### BACKGROUND INFORMATION

Certain methods for determining depths of focus have been described and are typically based on using a world camera with depth information. The use of a world camera can result in increased costs and increased energy consumption of smart glasses.

### SUMMARY

The present invention provides a method for determining an instantaneous depth of focus of a user of smart glasses, in particular at least by means of a single-eye tracking system. According to an example embodiment of the present invention, in at least one method step, an instantaneous Listing plane of a user eye is measured, wherein, in at least one further method step, an instantaneous vergence of the user eye is ascertained on the basis of the measured instantaneous Listing plane of the user eye, and wherein, in at least one additional further method step, the user-specific instantaneous depth of focus of the user eye is ascertained from the ascertained instantaneous vergence of the user eye. This can advantageously keep costs and/or energy consumption low, in particular since cost-intensive and/or energy-intensive use of a world camera can advantageously be dispensed with. Advantageously, a total number of components necessary for determining the instantaneous depth of focus in smart glasses, in particular smart glasses designed as a mono-eye system, can be kept low. Advantageously, the proposed method for determining the depth of focus can make possible applications based on the knowledge of the depth of focus, such as a focus-depth-adaptive manipulation of a display, in particular a virtual retinal scan display of smart glasses, a correction of the display, in particular of the virtual retinal scan display of the smart glasses, based on a current vergence of the user eye. In addition, a second measurement channel could advantageously be used in stereo systems and could, for example, be used to compensate for eye defects, such as nystagmus. In the case of a simple estimate of the intersection point of the gaze vectors ascertained from the stereo system, it would not be possible to take such eye defects into account.

The term “depth of focus of a user” is in particular understood to mean a distance of a focal point of the user eyes from the user eyes. In particular, the focal point of the user eyes is understood to mean the point in space in front of the user eyes where the gaze vectors of the user eyes intersect. The term “smart glasses” is in particular understood to mean a wearable (head-mounted display) by means of which information can be added to the field of view of a user. Smart glasses preferably make augmented-reality applications and/or mixed-reality applications possible. Smart glasses are also commonly referred to as VR glasses or AR glasses. In particular, the smart glasses comprise a virtual retinal scan display (also known as a light-field display), which is in particular generally conventional to a person skilled in the art. The virtual retinal scan display is in particular configured to image an image content, which has been scanned sequentially by deflecting at least one visible laser beam of at least one time-modulated light source, such as one or more (RGB) laser diodes of a laser projector, directly onto the retina of the eye of the user by means of optical elements. A single-eye tracking system is in particular configured to track and/or record a movement and/or a velocity of a single user eye. The terms “configured” and “provided” are in particular understood to mean specifically programmed, designed, and/or equipped. An object being provided and/or configured for a particular function is in particular understood to mean that the object fulfills and/or performs this particular function in at least one application state and/or operating state.

According to Listing's Law, all rotation axes of all saccades and of all slow subsequent movements of the user eye that originate from a primary position are located in one plane, the so-called Listing plane of the user eye. Not all eye positions possible in three-dimensional space, but only those that are limited to the rotation about rotation axes in the Listing plane are therefore taken when user eyes are fixed on distant objects. In particular, the Listing plane is formed as the plane in which, starting from the primary position of the user eye, all rotation axes of all possible eye positions achievable by rotation of the eyes are located. The location of the Listing plane is thus in particular dependent on the corresponding primary position. For example, in the case of a forward gaze (primary position=forward gaze), the Listing plane is approximately perpendicular to the optical axis of the user eye. For example, in the case of a forward gaze (primary position=forward gaze), the Listing plane corresponds to an equatorial plane of the user eye. A gaze shift from a starting position other than the primary position, e.g., an upward gaze by 30°, the rotation axes of the user eye are also located in a Listing plane, which, however, in comparison to the forward gaze, is no longer perpendicular to the gaze direction but is tilted in the direction of the gaze shift, in particular by a half angle of the gaze direction in relation to the forward-gaze primary position (here, by 15°). The Listing plane thus also tilts depending on the vergence, in particular proportionally to the depth of focus. Consequently, an instantaneous vergence of the user eye can advantageously be determined from the measured Listing plane. As a result, the vergence, in particular the tilting of the Listing plane during a change in eye focus, influences the exact movement trajectory, e.g., of the pupil, during the eye movement of the user eye. Knowledge of the vergence, in particular knowledge of the movement of two user eyes in opposite directions, preferably allows the intersection point of the gaze vectors and/or the instantaneous depth of focus of the user eye to be ascertained. The instantaneous vergence is in particular the instantaneous eye movement of the user eyes in opposite directions.

Furthermore, according to an example embodiment of the present invention, it is provided that, in the method step, the instantaneous Listing plane is ascertained at least on the basis of a measurement of an axis of rotation of a movement of the user eye, in particular by means of a laser feedback interferometry (LFI) sensor arrangement. In this way, costs and/or energy consumption can advantageously be kept low, in particular since cost-intensive and/or energy-intensive use of a world camera can advantageously be dispensed with. Advantageously, LFI sensors can be used to measure the depth of focus.

In particular, the LFI sensor arrangement comprises at least a plurality of, e.g., three or four, individual LFI sensors. Preferably, the LFI laser sensors of the LFI sensor arrangement are integrated into the smart glasses, e.g., into a frame of the smart glasses, into a glass lens of the smart glasses, and/or into a temple of the smart glasses. In particular, the LFI sensors of the LFI sensor arrangement are arranged in such a way that the laser beams, in particular the infrared laser beams, of the LFI sensors impinge on the user eye of the user wearing the smart glasses. If necessary, optical deflection elements can be provided, which deflect one or more of the laser beams onto the user eye. A conventional laser feedback interferometry measurement method that is made possible by the LFI sensors and may include modulation of the wavelengths of the lasers of the LFI sensors can advantageously measure a Doppler shift, which is due to a rotation of the corresponding user eye. In addition, a distance d between the particular user eye and the particular LFI sensor of the LFI sensor arrangement can advantageously be ascertained by means of the conventional LFI measurement method. Preferably, a surface velocity v of the user eye in a corresponding laser beam direction as well as the distance d can be ascertained for each of the LFI sensors of the LFI sensor arrangement from these measurement data of the LFI measurement method. Through a fusion of the measurement results of at least 3 LFI sensors of the LFI sensor arrangement, a rotational velocity of the user eye can thus in particular be ascertained. Through the fusion of the measurement results of at least 3 LFI sensors of the LFI sensor arrangement, a position of the user eye in relation to the smart glasses can thus in particular be ascertained. Furthermore, through the fusion of the measurement results of the LFI sensors of the LFI sensor arrangement, a current axis of rotation e can in particular be ascertained. In addition, through the fusion of the measurement results of the LFI sensors of the LFI sensor arrangement, a start gaze vector and/or a target gaze vector of the monitored eye movement can in particular be ascertained. For determining the start gaze vector and/or the target gaze vector, an eye-tracking camera, which is preferably provided for video-oculography, could alternatively also be integrated into the system. In this alternative case, a fusion of the measured values of the eye-tracking camera and of the (static) measured values of the LFI sensor arrangement can then be carried out.

In particular, for making the determination of the axis of rotation possible, in particular via the measurement data of the LFI sensor arrangement, a relation between the coordinate systems of the user eye and of the smart glasses must first be known (1st step of an algorithm for determining the axis of rotation). In particular, the positions and the orientations of the laser sensors in the coordinate system of the smart glasses are known from production, e.g., from an end-of-line measurement carried out in production or from an assembly accuracy required in production. In particular, the positions and orientations of the laser sensors in the coordinate system of the smart glasses can be assumed to be fixed. For determining a center of the user eye and/or of the coordinate system of the user eye, the user eye is preferably assumed to be a sphere of a defined and known diameter r. The relation between the coordinate system of the smart glasses and the coordinate system of the user eye is preferably expressed as a combination of a rotation and a translation. For ascertaining the relation between the coordinate system of the smart glasses and the coordinate system of the user eye, a triangulation approach is thus preferably used, see in particular equation (1).

$${x}_{g , n}^{T} {x}_{g , n} + 2 {x}_{g , n}^{T} {R}^{T} t + {t}^{T} t - {r}^{2} = 0 \qquad ( 1 )$$

In this case, x_{g} denotes a point in the coordinate system of the smart glasses, R denotes a rotation, t denotes a translation, and r denotes the radius of the user eye. The point x_{g} is in particular transformed into the coordinate system of the user eye via the rotation R and the translation t by taking into account the radius of the user eye, wherein the corresponding point is in particular denoted by xₕ. This results in equation (2)

$${x}_{h} = R ( θ , φ , r ) {x}_{g} + t \qquad ( 2 )$$

with the spherical coordinates θ, φ and r. The parameters R and t are preferably determined by means of a conventional trilateration method from the distance measurements of three LFI sensors of the LFI sensor arrangement.

After knowledge of the relation of the coordinate systems has been gained, surface velocities of the user eye at the impact points of the lasers of the LFI sensors are ascertained in a further step (2nd step of the algorithm for determining the axis of rotation). A surface velocity measured by an nth LFI sensor can in particular be ascertained from an impact point xₙ on the spherical surface of the user eye, from the previously ascertained orientation of the sensor (vector pₙ), and from the velocity vₙ measured via the Doppler shift, see equation (3).

$${v}_{n} = - {p}_{n}^{T} ( \frac{\partial {x}_{n}}{\partial r} \dot{r} + \frac{\partial {x}_{n}}{\partial θ} \dot{θ} + \frac{\partial {x}_{n}}{\partial φ} \dot{φ} ) = - {p}_{n}^{T} ( \frac{{x}_{n}}{‖ {x}_{n} ‖} \dot{r} + \frac{\partial {x}_{n}}{\partial θ} + \frac{\partial {x}_{n}}{\partial φ} ) \qquad ( 3 )$$

Here, the surface velocities are implemented as the changes in the angular velocities about the rotation axis of the eye. In particular, a change in radius, i.e., a displacement of the impact point, can lead to a change in velocity that must be compensated (however, this does not apply to the retina since rotation of a sphere does not cause a change in the radius).

This in particular allows the equation system (4) including the rotation axis e and the angular velocities \dot{θ} and \dot{φ} to be set up.

$$[ - {p}_{n}^{T} ( {e}_{θ} \times {x}_{n} ) - {p}_{n}^{T} ( {e}_{φ} \times {x}_{n} ) ] [ \begin{matrix}\dot{θ} \\ \dot{φ}\end{matrix} ] = {v}_{n} - \frac{{x}_{n}}{‖ {x}_{n} ‖} {\dot{r}}_{n} \qquad ( 4 )$$

Only the measured values of the velocity vₙ measured by the nth LFI sensor at its impact point on the user eye as well as the ascertained impact point xₙ can be used in the equation system (4). This equation system (4) can then be solved for the unknown axis of rotation e. If an integration with respect to the angular velocity is now carried out, a rotation of the user eye, in particular a high-resolution movement trajectory of the user eye (θ, φ) along the rotation axis e of the user eye can be determined, in particular exclusively from the measurements of the LFI sensor arrangement.

According to an example embodiment of the present invention, it is also provided that, in the method step, the instantaneous Listing plane is ascertained at least on the basis of a measurement of a start position of a movement of the user eye about the axis of rotation and/or on the basis of a measurement of an end position of a movement of the user eye about the axis of rotation, in particular by means of a laser feedback interferometry (LFI) sensor arrangement of the smart glasses or by means of an eye-tracking camera of the smart glasses, (3rd step of the algorithm for determining the axis of rotation). In this way, costs and/or energy consumption can advantageously be kept low, in particular since cost-intensive and/or energy-intensive use of a world camera can advantageously be dispensed with. Advantageously, it is possible to use only the LFI sensors to measure the start position and/or the end position. Alternatively, however, determining the start position and/or the end position by means of the eye-tracking camera and video-oculography is also possible, which, however, would not fully realize the described advantages. The movement of the user eye associated with the start position and the end position can be carried out intentionally or by a saccade.

In particular, for determining the (absolute) start position and/or the end position of the gaze vector, a triangulation of an iris plane of an iris of the user eye in space is carried out during the movement along the movement trajectory of the user eye. In particular, at least the laser beams from three of the LFI sensors must impinge on the iris of the user eye for this purpose. In particular, an absolute orientation of the iris of the user eye can then be ascertained. The iris is preferably assumed to be disk-shaped in the underlying model (“iris disk”). Equations (5) to (7) can then in particular be used to determine absolute gaze angles θₐ and φ₀.

$${n}_{Iris}^{T} x = {d}_{Iris} \qquad ( 5 )$$

$${θ}_{0} = arc sin \frac{{n}_{Iris , z}}{‖ n ‖} \qquad ( 6 )$$

$${φ}_{0} = arc tan \frac{- {n}_{Iris , x}}{{n}_{Iris , y}} \qquad ( 7 )$$

A normal vector n_{Iris} that points in the gaze direction then preferably results from a reconstructed plane that can be generated from the triangulation of the distance measurements of the three LFI sensors whose laser beams impinged on the iris of the user eye. Alternatively, the absolute position of the user eye can also be ascertained from the optionally additionally installed eye-tracking camera with the aid of video-oculography algorithms. From the three described steps of the algorithm for determining the axis of rotation, at least the following parameters thus result: a) movement trajectory θ(t), φ(t) of the user eye during an eye movement, b) axis of rotation e about which the user eye has rotated during the eye movement, and c) absolute position of the user eye (θ₀, φ₀) at a beginning and at an end of the eye movement.

Furthermore, according to an example embodiment of the present invention, it is provided that, in the method step, the movement trajectory of the movement of the user eye is ascertained from the detected axis of rotation of the movement of the user eye and from the detected start and end positions of the movement of the user eye. This can advantageously make it possible to determine the Listing plane of the user eye, and thus in particular also the depth of focus of the user eye, by means of a system that incurs particularly low costs and/or energy consumptions.

In particular, the movement trajectories form orthodromes of the corresponding associated Listing planes or half-angle planes.

In addition, according to an example embodiment of the present invention, it is provided that, in the method step, the surface velocity, in particular a rotational surface velocity, of the user eye is detected by means of the LFI sensor arrangement of the smart glasses, preferably in the manner described above (cf. especially equation (3)), and is evaluated for determining the axis of rotation and/or the movement trajectory, in particular in the manner described (cf. especially equation (4)).

This can advantageously make it possible to determine the Listing plane of the user eye, and thus in particular also the depth of focus of the user eye, by means of a system that incurs particularly low costs and/or energy consumptions.

Furthermore, according to an example embodiment of the present invention, it is provided that, in the further method step, the instantaneous vergence is ascertained on the basis of a Listing plane angle between the measured instantaneous Listing plane and a (non-tilted) Listing plane, which is in particular initially determined and/or calibrated, for a primary position of a user eye gazing into infinity. This can advantageously make it possible to determine the depth of focus of the user eye by means of a system that incurs particularly low costs and/or energy consumptions. In particular, a location of the primary position is determined by an initial calibration. In particular, a relationship between the location of the instantaneous Listing plane and the vergence is determined by an initial calibration. In particular, for a given start position, a set of half-angle planes may be given, which each follow from a particular rotation of the Listing plane. In particular, if the axis of rotation is known, the half-angle plane, and thus the location of the instantaneous Listing plane or the actual primary position, can generally be unambiguously ascertained from the set of half-angle planes that contains this axis of rotation. Furthermore, according to an example embodiment of the present invention, it is provided that, in the further method step, the instantaneous vergence is read from a known, in particular previously calibrated, characteristic curve or look-up table, in which vergences are plotted over Listing plane angles. This can advantageously make it possible to determine the depth of focus of the user eye by means of a system that incurs particularly low costs and/or energy consumptions. In particular, the characteristic curve or the lookup table describes the relationship (to be calibrated, if necessary) between the instantaneous Listing plane of the user eye and the current vergence of the user eye. In this context, it is noted as an aside that, if the vergence is known (e.g., by measuring both eyes simultaneously), this information could be used in the reverse as a restriction for determining the axis of rotation of the user eye.

In addition, according to an example embodiment of the present invention, in the additional further method step, the user-specific instantaneous depth of focus of the user eye is ascertained using initial user calibration data, which link different vergences to different user-specific depths of focus of the user eye. This can advantageously make it possible to determine the depth of focus of the user eye by means of a system that incurs particularly low costs and/or energy consumptions. It can be seen from the above-described portions of the method that the vergence of the user eye can be ascertained from the measured movement trajectory, the start and target positions of the gaze vector, and the axis of rotation. In order finally to be able to transform this into a depth of focus of the user eyes, a (one-time) system calibration (per user) is in particular required, in particular since the smart glasses can sit differently on a user face depending on the user, i.e., the coordinate system of smart glasses—user head can be different (e.g., depending on the head shape, nose shape, pupil spacing, etc.).

In this context, according to an example embodiment of the present invention, it is provided that, in a user calibration step temporally preceding the additional further method step, the initial user calibration data can be ascertained by tracking at least one moved real-world calibration object by means of a world camera of the smart glasses and simultaneously measuring, by means of a LFI sensor arrangement of the smart glasses or by means of an eye-tracking camera of the smart glasses, the vergence of the user eye following the moved real-world calibration object and focusing on the moved real-world calibration object, in particular by evaluating known geometric parameters of the moved real-world calibration object at various locations and distances of the moved real-world calibration object from the world camera and simultaneously assigning them to the measured vergences, and/or by ascertaining various locations and distances of the moved real-world calibration object from the world camera by means of a stereo system and/or a structured light system and simultaneously assigning them to the measured vergences. This can advantageously make it possible to determine the depth of focus for smart glasses. The movable real-world calibration object can in particular be a finger, e.g., an index finger, of a user hand. For example, the user extends his hand and points upward with the index finger for this purpose. In this example, the fingertip can then be detected by the world camera, and a depth of focus between the world camera (which is fixed in the coordinate system of the smart glasses) and the user eye or smart glasses can be ascertained from geometrical conditions of the fingertip (e.g., a shape or a thickness of the finger or of the fingernail of the finger) or by means of a stereo system or a structured light system of the smart glasses (e.g., multiple controllable light sources arranged at different points of the smart glasses). In this example, the user can subsequently slowly move the finger in the direction of the world camera toward the smart glasses while the user eyes are fixed on the finger. For example, prompted by an information output of a display of the smart glasses or by an acoustic signal of the smart glasses, the user stops moving the finger toward the smart glasses at a particular depth of focus and then moves the finger in the corresponding depth of focus in parallel with the user head to the left and right, in particular in order to cause so-called “smooth pursuit eye movements” of the user eye at the corresponding depth of focus. These movements of the user eye can then be detected and/or recognized by the smart glasses, and corresponding depth information from the world camera can then be mapped to the position of the displaced instantaneous Listing plane, in particular in comparison to the primary position of the user eye. This process is preferably repeated until sufficient calibration points have been collected (for example, at least two calibration points are necessary for a linear 2-point calibration). The real-world calibration object may be the finger as described or else another object, preferably an object whose dimensions are known, for example. In particular, the user calibration step also includes a measurement with eyes gazing into infinity, i.e., with a vergence of zero.

The term “world camera” is in particular understood to mean a camera of the smart glasses that is aligned in a direction facing away from the user face. Preferably, a field of view of the world camera of the smart glasses overlaps at least to a large extent with a field of view of the user who looks through the glass lenses of the smart glasses.

In addition, according to an example embodiment of the present invention, it is provided that, in the user calibration step, the real-world calibration object is moved at various (e.g., two, three or four) distances from the world camera, in each case in distance planes at least substantially parallel to an image plane of the world camera. This can advantageously cause the described “smooth pursuit eye movements” of the user eye at the depths of focus corresponding to the different distances. As a result, the depth information from the world camera can advantageously be mapped to the position of the displaced instantaneous Listing plane, in particular in comparison to the primary position of the user eye. The image plane of the world plane is in particular aligned at least substantially perpendicularly to a gaze direction of the user eye in the primary position. The term “substantially (in) parallel” is in particular understood to mean an alignment of a direction relative to a reference direction, in particular in a plane, wherein the direction has a deviation from the reference direction of in particular less than 8°, advantageously less than 5°, and particularly advantageously less than 2°. The term “substantially perpendicular(ly)” here is in particular to define an alignment of a direction relative to a reference direction, wherein the direction and the reference direction, in particular as viewed in a projection plane, enclose an angle of 90°, and the angle has a maximum deviation of in particular less than 8°, advantageously less than 5°, and particularly advantageously less than 2°.

In addition, in accordance with an example embodiment of the present invention, it is also provided that the movement of the real-world calibration object in the user calibration step is carried out manually by the user, wherein the movement of the real-world calibration object is monitored by the world camera, and movement specifications and/or movement commands are ascertained on the basis of the monitoring data acquired in the process and are output acoustically and/or visually to the user by the smart glasses. This can advantageously make simple and reliable initial user calibration possible. The movement specifications and/or movement commands can in particular be output visually by means of the virtual retinal scan display of the smart glasses.

Alternatively or additionally, according to an example embodiment of the present invention, it is provided that, in an alternative or additional user calibration step temporally preceding the additional further method step, the initial user calibration data are ascertained by showing a moved virtual calibration object by means of a display of the smart glasses, in particular by means of the virtual retinal scan display of the smart glasses, and by simultaneously measuring, by means of the LFI sensor arrangement of the smart glasses or by means of the eye-tracking camera of the smart glasses, the vergence of the user eye following the moved virtual calibration object and focusing on the moved virtual calibration object. This can advantageously make it possible to determine the depth of focus for smart glasses. In addition, this can advantageously make it possible to determine the depth of focus of the user eye by means of a system that incurs particularly low costs and/or energy consumptions and in particular does not require a world camera. For example, a stimulus (e.g., a moving point, a text, etc.) can be shown virtually in various focal planes for this purpose, e.g., by means of the virtual retinal scan display of the smart glasses, and the corresponding eye movements can again be measured via the system of the smart glasses, in particular via the LFI sensor arrangement of the smart glasses. This can advantageously make it possible to map the instantaneous (displaced) Listing plane in relation to the primary position to the corresponding focal plane. This design is particularly advantageous since it makes direct calibration of the depth of focus to display contents of the virtual retinal scan display of the smart glasses possible.

If, as mentioned above, in the alternative or additional user calibration step, the virtual calibration object is shown and moved by means of the display of the smart glasses, in particular by the virtual retinal scan display, at various focal distances, in particular generated by an optical system of the smart glasses, e.g., a lens system of the smart glasses or the laser projector of the smart glasses, direct calibration of the depth of focus can advantageously be made possible on the basis of display contents of the virtual retinal scan display of the smart glasses and/or for display contents of the virtual retinal scan display of the smart glasses.

In addition, a computer unit for determining the instantaneous depth of focus of the user of the smart glasses is proposed, wherein the computer unit is at least configured to ascertain the instantaneous vergence of the user eye on the basis of an instantaneous Listing plane of the user eye, which Listing plane is in particular measured at least by means of the single-eye tracking system, and wherein the computer unit is at least configured to ascertain the user-specific instantaneous depth of focus of the user eye from the ascertained instantaneous vergence of the user eye. This can advantageously keep costs and/or energy consumption low. Advantageously, a total number of components necessary for determining the instantaneous depth of focus in smart glasses, in particular smart glasses designed as a mono-eye system, can be kept low. The computer unit can in particular be microcontrollers or the like of the smart glasses. In this case, the computer unit can in particular be understood as a unit with at least one electronic control system. The term “electronic control system” is in particular understood to mean a unit with a processor and a storage medium as well as an operating program stored in the memory unit. Alternatively, the computer unit may also be at least partially formed by a cloud or another central computing infrastructure (e.g., a smartphone coupled to the smart glasses) or decentralized computing infrastructure separate from the smart glasses. In this case, the smart glasses preferably comprise a (wireless) communication interface for communication with the external part of the computer unit.

Furthermore, smart glasses with at least one internal or external computer unit and with a laser feedback interferometry (LFI) sensor arrangement are provided by the present invention, wherein the smart glasses, in particular the computer unit, are configured at least to determine the instantaneous depth of focus of the user of the smart glasses. This can advantageously keep costs and/or energy consumption low, in particular since cost-intensive and/or energy-intensive use of a world camera can advantageously be dispensed with. Advantageously, a total number of components necessary for determining the instantaneous depth of focus in smart glasses, in particular smart glasses designed as a mono-eye system, can be kept low.

The method according to the present invention and the smart glasses according to the present invention are not to be limited to the application and embodiments described above. In particular, for fulfilling a described functionality, the method according to the present invention and/or the smart glasses according to the present invention can comprise a number of individual elements, components, units, and method steps that deviates from a number mentioned here. In addition, for the value ranges specified in this disclosure, values within the mentioned limits are also to be considered disclosed and usable as desired.

### BRIEF DESCRIPTION OF THE DRAWINGS

Further advantages of the present invention result from the following description of the figures. An exemplary embodiment of the present invention is illustrated in the figures. The disclosure herein contains numerous features in combination. A person skilled in the art will expediently also consider the features individually and combine them to form meaningful further combinations, in view of the disclosure herein.

FIG. 1 shows a schematic partial view of smart glasses, according to an example embodiment of the present invention.

FIG. 2 shows a diagram of a vergence of user eyes of a user of the smart glasses, according to an example embodiment of the present invention.

FIG. 3 shows a schematic flow diagram of a method for determining an instantaneous depth of focus of the user of the smart glasses, according to an example embodiment of the present invention.

FIG. 4 shows a schematic side view of the smart glasses and of a real-world calibration object during a user calibration step of the method, according to an example embodiment of the present invention.

FIG. 5A shows a diagram of the user eye gazing into infinity and of an associated (non-tilted) Listing plane, according to an example embodiment of the present invention.

FIG. 5B shows a diagram of an instantaneous Listing plane of a user eye not gazing into infinity, i.e., a focused user eye, according to an example embodiment of the present invention.

FIG. 6 shows a diagram of exemplary movement trajectories of user eyes with different primary positions, according to an example embodiment of the present invention.

### DETAILED DESCRIPTION OF EXAMPLE EMBODIMENTS

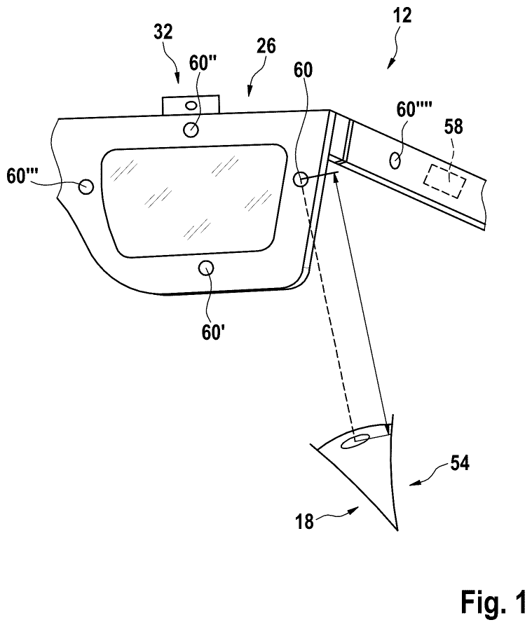

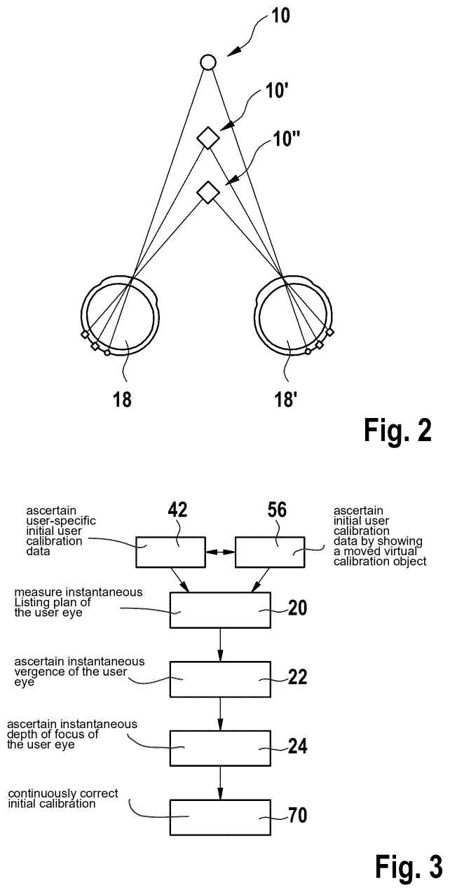

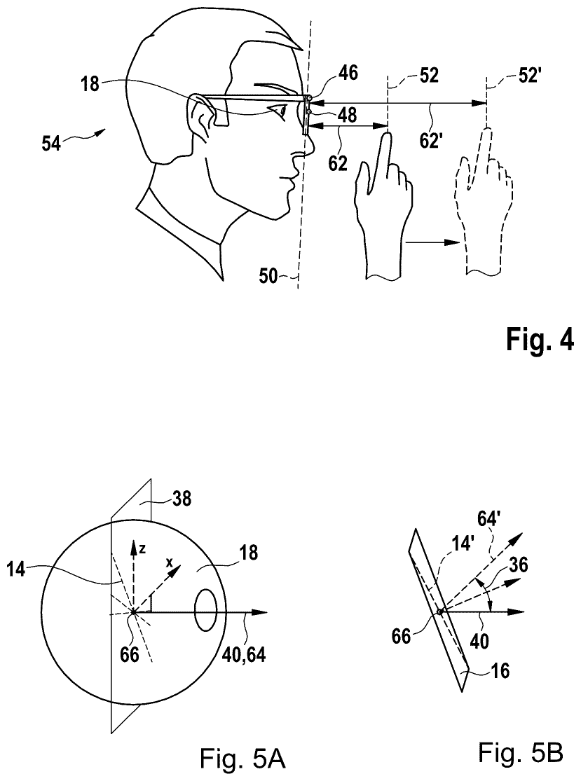

FIG. 1 shows a schematic partial view of smart glasses 12. The smart glasses 12 comprise a virtual retinal scan display for a visual representation of virtual contents, e.g., augmented-reality contents, in a field of view of a user 54 of the smart glasses 12. The basic function and basic components of the smart glasses 12 are comparable to those of conventional smart glasses 12 with virtual retinal scan displays. The smart glasses 12 comprise a single-eye tracking system (mono-eye tracking system). The smart glasses 12 comprise a computer unit 58. By way of example, the computer unit 58 is designed as an internal computer unit. Alternatively, however, the computer unit 58 could also be designed to be at least partially separate from the smart glasses 12 (e.g., as a mobile end device, such as a smartphone or the like, coupled to the smart glasses 12). The smart glasses 12 comprise a laser feedback interferometry (LFI) sensor arrangement 26. The LFI sensor arrangement 26 comprises, by way of example, five LFI sensors 60, 60′, 60″, 60″′, 60″″. Alternatively, however, more or less than five LFI sensors 60, 60′, 60″, 60″′, 60″″ are also possible. The smart glasses 12 comprise an eye-tracking camera 32. The eye-tracking camera 32 has a field of view aligned in the direction of a user eye 18 of the user 54. The eye-tracking camera 32 is configured to track at least one user eye 18, in particular its position in a coordinate system of the smart glasses 12. The smart glasses 12, in particular the computer unit 58, are configured to determine an instantaneous depth of focus 10 (cf. FIG. 2) of the user 54 of the smart glasses 12. The smart glasses 12 are configured to perform a method for determining the instantaneous depth of focus 10 of the user 54 of the smart glasses 12. The smart glasses 12 may comprise a world camera 46 (see FIG. 4) or can be formed free of a world camera.

FIG. 2 schematically shows a vergence of the user eyes 18, 18′ of the user 54 of the smart glasses 12. In FIG. 2, the user eyes 18, 18′ are shown at three different depths of focus 10, 10′, 10″ by way of example. As the depth of focus 10, 10′, 10″ decreases, both gaze directions of both user eyes 18, 18′ move toward each other. As the depth of focus 10, 10′, 10″ increases, both gaze directions of both user eyes 18, 18′ move away from each other. Each depth of focus 10, 10′, 10″ is assigned a focal point (marked by a circle or diamond in FIG. 2).

FIG. 3 shows a schematic flow diagram of the method for determining the instantaneous depth of focus 10, 10′, 10″ of the user 54 of the smart glasses 12. The computer unit 58, in particular the smart glasses 12, is configured to perform the method for determining the instantaneous depth of focus 10, 10′, 10″ of the user 54. In at least one user calibration step 42, user-specific initial user calibration data are ascertained. The user calibration data are ascertained by tracking at least one moved real-world calibration object 44 (cf. FIG. 4) by means of a world camera 46 of the smart glasses 12 and simultaneously measuring, by means of the LFI sensor arrangement 26 of the smart glasses 12 or by means of the eye-tracking camera 32 of the smart glasses 12, the vergence of the user eye 18, 18′ following the moved real-world calibration object 44 and focusing on the moved real-world calibration object 44. By way of example, the real-world calibration object 44 is shown as a finger of the user 54 in FIG. 4. For ascertaining the user calibration data, in particular a user-specific vergence behavior, in the user calibration step 42, known geometric parameters of the moved real-world calibration object 44 at various locations and distances 62, 62′ of the moved real-world calibration object 44 from the world camera 46 are evaluated and simultaneously assigned to the measured vergences. Alternatively, various locations and distances 62, 62′ of the moved real-world calibration object 44 from the world camera 46 could be ascertained by means of a stereo system and/or a structured light system 48 of the smart glasses 12 and simultaneously assigned to the measured vergences. In the example of the finger, the known geometric parameter is a width of a fingernail of the finger. In the user calibration step 42, the real-world calibration object 44 is also moved at the various distances 62, 62′ from the world camera 46, in each case in a distance plane 52 at least substantially parallel to an image plane 50 of the world camera 46. The movement of the real-world calibration object 44 in the user calibration step 42 is carried out manually by the user 54. In the user calibration step 42, the movement of the real-world calibration object 44 is also monitored by the world camera 46, and movement specifications and/or movement commands are ascertained on the basis of the monitoring data acquired in the process. In the user calibration step 42, the ascertained movement commands are also output acoustically and/or visually to the user 54 by the smart glasses 12.

In at least one alternative or additional user calibration step 56, the initial user calibration data are ascertained by showing a moved virtual calibration object by means of the virtual retinal scan display of the smart glasses 12, and by simultaneously measuring, by means of the LFI sensor arrangement 26 of the smart glasses 12 or by means of the eye-tracking camera 32 of the smart glasses 12, the vergence of the user eye 18, 18′ following the moved virtual calibration object 44 and focusing on the moved virtual calibration object 44. In the alternative user calibration step 56, the virtual calibration object is shown and moved by the display of the smart glasses 12 at various focal distances generated, for example, by an optical system (not shown) of the smart glasses 12.

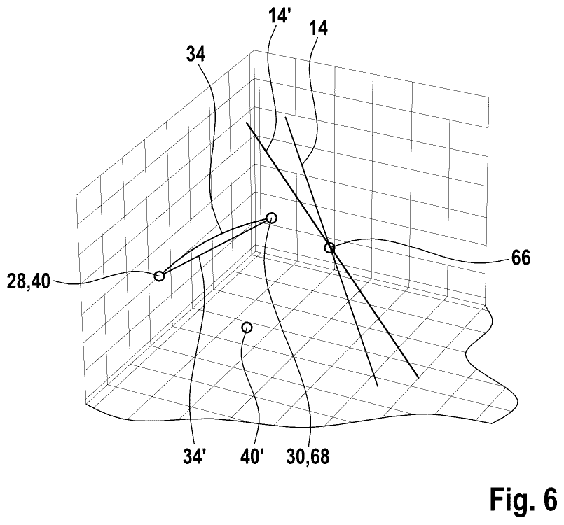

In at least one method step 20, an instantaneous Listing plane 16 (cf. FIG. 5b) of the user eye 18 is measured. In the method step 20, the instantaneous Listing plane 16 is ascertained at least on the basis of a measurement of an axis of rotation 14 (cf. FIGS. 5A and 5B) of a movement of the user eye 18 by means of the LFI sensor arrangement 26. In the method step 20, a surface velocity of the user eye 18 is detected by means of the LFI sensor arrangement 26 of the smart glasses 12 and evaluated for determining the axis of rotation 14. In the method step 20, the instantaneous Listing plane 16 is ascertained at least on the basis of a measurement of a start position 28 (cf. FIG. 6) of a movement of the user eye 18 about the axis of rotation 14 by means of the LFI sensor arrangement 26 of the smart glasses 12 or by means of the eye-tracking camera 32 of the smart glasses 12. In the method step 20, the instantaneous Listing plane 16 is ascertained at least on the basis of a measurement of an end position 30 (cf. FIG. 6) of a movement of the user eye 18 about the axis of rotation 14 by means of the LFI sensor arrangement 26 of the smart glasses 12 or by means of the eye-tracking camera 32 of the smart glasses 12. In the method step 20, a movement trajectory 34, 34′ (cf. FIG. 6) of the movement of the user eye 18 is ascertained from the detected axis of rotation 14 of the movement of the user eye 18 and from the detected start and end positions 28, 30 of the movement of the user eye 18. In the method step 20, the surface velocity of the user eye 18 is detected by means of the LFI sensor arrangement 26 of the smart glasses 12 and evaluated for determining the movement trajectory 34, 34′.

In at least one further method step 22, an instantaneous vergence of the user eye 18 is ascertained on the basis of the measured instantaneous Listing plane 16 of the user eye 18. In the further method step 22, the instantaneous vergence is ascertained on the basis of a Listing plane angle 36 (cf. FIG. 5b) between the measured instantaneous Listing plane 16 and an initially determined and/or calibrated Listing plane 38 for a primary position 40 of the user eye 18 gazing into infinity. In the further method step 22, the instantaneous vergence is read from a known, previously calibrated characteristic curve, in which vergences are plotted over Listing plane angles 36.

In at least one additional further method step 24, the user-specific, instantaneous depth of focus 10, 10′, 10″ of the user eye 18 is ascertained from the ascertained instantaneous vergence of the user eye 18. In the additional further method step 24, the user-specific instantaneous depth of focus 10, 10′, 10″ of the user eye 18 is ascertained using the user calibration data ascertained in one of the user calibration steps 42, 56 or the user calibration data ascertained in the two initial user calibration steps 42, 56, which user calibration data links different vergences to different user-specific depths of focus 10, 10′, 10″ of the user eye 18.

In at least one further method step 70, the initial calibration from the user calibration steps 42, 56 is continuously corrected to the setpoint. This is possible, for example, during reading of contents in various distance planes, 52, 52′, in order, for example, advantageously to make it possible to correct the calibration function to the setpoint when the smart glasses 12 slip. Possible applications of the above-described method are, for example, manipulation of a user interface (UI) of the smart glasses 12 in a single-eye tracking system, redundant measurements of the depths of focus 10 in the stereo system for taking into account refractive errors, such as nystagmus or squints, or adaptation of the virtual retinal scan display of the smart glasses 12, e.g., of a VR headset, to the eye position (misalignment of the eyes).

FIG. 4 shows a schematic side view of the smart glasses 12 and of the real-world calibration object 44 during the user calibration step 42. By way of example, two different distances 62, 62′ of the real-world calibration object 44 from the smart glasses 12 are shown.

FIG. 5A schematically shows the user eye 18 gazing into infinity and the associated (non-tilted) Listing plane 38. All possible axes of rotation 14 of the user eye 18 for movements of the user eye 18 from the eye position gazing into infinity are located in the Listing plane 38. In FIG. 5A, the user eye 18 is in the primary position 40. The primary position 40 is characterized by the vector drawn in FIG. 5A and perpendicular to the Listing plane 38. When gazing into infinity, the vector corresponds to the primary position 40 of a gaze direction 64 of the user eye 18.

FIG. 5B schematically shows an instantaneous Listing plane 16 of a user eye 18 not gazing into infinity, i.e., a focused user eye 18. The instantaneous Listing plane 16 is tilted in comparison to the Listing plane 38 of the eye 18 gazing into infinity. A tilt angle of the tilt of the Listing plane 16 in comparison to the Listing plane 38 of the primary position 40 is half the angle between the gaze direction 64′ of the focused eye 18 and the primary position 40 (the vector characterizing the primary position 40). All possible axes of rotation 14′ of eye movements originating from this point are located in the instantaneous Listing plane 16 of the focused user eye 18. The Listing plane 16 of the focused user eye 18 forms a half-angle plane of the angle between the gaze direction 64′ of the focused user eye 18 and the gaze direction 64 of the user eye 18 gazing into infinity.

FIG. 6 schematically illustrates exemplary movement trajectories 34, 34′ of user eyes 18 having different primary positions 40, 40′. The axes of rotation 14, 14′ are different depending on the associated primary position 40, 40′. The movement trajectories 34, 34′ are different depending on the associated primary position 40, 40′. In the case shown, the eye center points 66 of the two primary positions 40, 40′ are located on top of each other. The rotations of the associated user eyes 18 occur about the eye center point 66 in each case. In the case of the first movement trajectory 34, the primary position 40 corresponds to the starting point of the movement along the movement trajectory 34 and the gaze direction 64 of the user eye 18 gazing into infinity. The movement trajectory 34 to the target point 68 of the movement of the user eye 18 follows from the associated axis of rotation 14. In this case, the movement trajectory 34 corresponds to the orthodromes of the user eye 18 assumed to be spherical. In the case of the second movement trajectory 34′, the primary position 40′ does not correspond to the starting point of the movement along the movement trajectory 34′. However, the starting point of the movement along the two movement trajectories 34, 34′ shown is identical. In the case of the second movement trajectory 34′, the axis of rotation 14′ is located in the half-angle plane (angle-bisecting plane) between the primary position 40′ and the starting point of the movement along the second movement trajectory 34′. Accordingly, the second movement trajectory 34′ describes a different path to reach the same target point 68 as the first movement trajectory 34. The movement trajectory 34, 34′ for the same starting points and the same target points 68 thus changes depending on the vergence of the user eyes 18 and thus the primary positions 40, 40′.

1-15. (canceled)

16. A method for determining an instantaneous depth of focus of a user of smart glasses using a single-eye tracking system, the method comprising the following steps:
measuring an instantaneous Listing plane of a user eye;
ascertaining an instantaneous vergence of the user eye based on the measured instantaneous Listing plane of the user eye; and
ascertaining a user-specific instantaneous depth of focus of the user eye from the ascertained instantaneous vergence of the user eye.

17. The method according to claim 16, wherein the instantaneous Listing plane is ascertained at least based on a measurement of an axis of rotation of a movement of the user eye using a laser feedback interferometry sensor arrangement.

18. The method according to claim 16, wherein the instantaneous Listing plane is ascertained at least based on a measurement of a start position of a movement of the user eye about an axis of rotation and/or based on a measurement of an end position of a movement of the user eye about the axis of rotation, using a laser feedback interferometry (LFI) sensor arrangement of the smart glasses or using an eye-tracking camera of the smart glasses.

19. The method according to claim 18, wherein a movement trajectory of the movement of the user eye is ascertained from the detected axis of rotation of the movement of the user eye and from the detected start and end positions of the movement of the user eye.

20. The method according to claim 18, wherein a surface rotational velocity of the user eye is detected using the LFI sensor arrangement of the smart glasses and evaluated for determining the axis of rotation and/or a movement trajectory.

21. The method according to claim 16, wherein the instantaneous vergence is ascertained based on a Listing plane angle between the measured instantaneous Listing plane and a Listing plane, which is initially determined and/or calibrated, for a primary position of a user eye gazing into infinity.

22. The method according to claim 21, wherein, the instantaneous vergence is read from a known, previously calibrated, characteristic curve, in which vergences are plotted over Listing plane angles.

23. The method according to claim 16, wherein, in an the user-specific instantaneous depth of focus of the user eye is ascertained using initial user calibration data, which link different vergences to different user-specific depths of focus of the user eye.

24. The method according to claim 23, wherein, in a user calibration step, temporally preceding the ascertaining of the user-specific instantaneous depth of focus of the user eye, the initial user calibration data are ascertained by tracking at least one moved real-world calibration object using a world camera of the smart glasses and simultaneously measuring, using a laser feedback interferometry (LFI) sensor arrangement of the smart glasses or using an eye-tracking camera of the smart glasses, a vergence of the user eye following the moved real-world calibration object and focusing on the moved real-world calibration object: (i) by evaluating known geometric parameters of the moved real-world calibration object at various locations and distances of the moved real-world calibration object from the world camera and simultaneously assigning them to the measured vergences, and/or (ii) by ascertaining various locations and distances of the moved real-world calibration object from the world camera using a stereo system and/or a structured light system and simultaneously assigning them to the measured vergences.

25. The method according to claim 24, wherein the real-world calibration object is moved at various distances from the world camera, in each case in a distance plane at least substantially parallel to an image plane of the world camera.

26. The method according to claim 25, wherein the movement of the real-world calibration object is carried out manually by the user, wherein the movement of the real-world calibration object is monitored by the world camera, and movement specifications and/or movement commands are ascertained based on monitoring data acquired during the movement and are output acoustically and/or visually to the user by the smart glasses.

27. The method according to claim 23, in a user calibration step, temporally preceding the ascertaining of the user-specific instantaneous depth of focus of the user eye, initial user calibration data are ascertained by showing a moved virtual calibration object using a display of the smart glasses, and by simultaneously measuring, using a laser feedback interferometry (LFI) sensor arrangement of the smart glasses or using eye-tracking camera of the smart glasses, the vergence of the user eye following the moved virtual calibration object and focusing on the moved virtual calibration object.

28. The method according to claim 27, wherein the virtual calibration object is shown and moved by the display of the smart glasses at various focal distances, generated by an optical system of the smart glasses.

29. A computer unit for determining an instantaneous depth of focus of a user of smart glasses, wherein the computer unit is configured at least to ascertain an instantaneous vergence of the user eye based on an instantaneous Listing plane of the user eye, the instantaneous Listing plane being measured at least using a single-eye tracking system, and wherein the computer unit is at least configured to ascertain the user-specific instantaneous depth of focus of the user eye from the ascertained instantaneous vergence of the user eye.

30. Smart glasses, comprising:
at least one internal or external computer unit; and
a laser feedback interferometry (LFI) sensor arrangement;
wherein the computer unit is configured to determine an instantaneous depth of focus of a user of the smart glasses by:
measuring an instantaneous Listing plane of a user eye,
ascertaining an instantaneous vergence of the user eye based on the measured instantaneous Listing plane of the user eye, and
ascertaining a user-specific instantaneous depth of focus of the user eye from the ascertained instantaneous vergence of the user eye.
